(19) United States
(12) Patent Application Publication
(10) Pub. No.: US 2016/0256325 A1
(43) Pub. Date: Sep. 8, 2016

(54) CONTROL DEVICE FOR A LASER SYSTEM, LASER SYSTEM, AND METHOD FOR CONTROLLING THE LASER SYSTEM

(71) Applicant: CARL ZEISS MEDITEC AG, Jena (DE)

(72) Inventors: Delbert Peter ANDREWS, Oberkochen (DE); Michael Stefan RILL, Jena (DE); Tobias DAMM, Munich (DE); Ekkehard FABIAN, Rosenheim (DE)

(21) Appl. No.: 15/025,567

(22) PCT Filed: Sep. 30, 2014
(86) PCT No.: PCT/EP2014/002660

§ 371 Date: Mar. 29, 2016

(30) Foreign Application Priority Data
Sep. 30, 2013 (DE) 10 2013 016 333.1

(51) Int. Cl. A61F 9/008

(52) U.S. Cl. CPC A61F 9/00825; A61F 9/0084; A61F 2009/00887; A61F 9/00745

(57) ABSTRACT

A control device for a laser system having a laser for introducing cuts into a lens of a human or animal eye configured to arrange and move a focal point of a laser beam of the laser within the lens, wherein the focal point can be moved for at least one cut for the fragmentation or segmentation of the lens into individual fragments or segments and can be moved according to a definable cutting pattern for the fine fragmentation of the lens, determine an individual one of the segments, and move, for the fine fragmentation within the determined segment, the focal point in a radial direction at most up to a distance greater than zero from an edge of the lens.

### CROSS REFERENCE TO RELATED APPLICATIONS

This application is a U.S. National Stage Application under 35 U.S.C. §371 of International Application No. PCT/EP2014/002660 filed on Sep. 30, 2014, and claims benefit to German Patent Application No. DE 10 2013 016 333.1 filed on Sep. 30, 2013. The International Application was published in German on Apr. 2, 2015 as WO 2015/043770 A1 under PCT Article 21(2).

### FIELD

The invention relates to a control device for a laser system with a laser for introducing cuts into a lens of a human or animal eye. The invention further relates to a laser system including such a control device and a method for controlling such a laser system.

### BACKGROUND

Medical laser systems can be used to introduce cuts into eye tissue, for example, lens tissue affected by cataract. Laser cuts make it possible to at least partially dispense with the phacoemulsification (destruction of the lenses by means of an ultrasonic transducer), which is risky for a patient, because the lens can be dissected or finely fragmented into small individual parts by means of the laser. Depending on experience or the attributes of a respective lens, a surgeon can segment the lens nucleus either only into rough parts (in particular in the shape of cake slices) or additionally finely fragment it into several small parts (e.g. in the shape of a cube).

US 2012/0259320 A1 describes a device for fragmenting a lens, by way of which rough cuts can be introduced into the lens in order to subsequently carry out a fine fragmentation of the lens.

### SUMMARY

In an embodiment, the present invention provides a control device for a laser system having a laser for introducing cuts into a lens of a human or animal eye. The control device is configured to arrange and move a focal point of a laser beam of the laser within the lens, wherein the focal point can be moved for at least one cut for the fragmentation or segmentation of the lens into individual fragments or segments and can be moved according to a definable cutting pattern for the fine fragmentation of the lens, determine an individual one of the segments, and move, for the fine fragmentation within the determined segment, the focal point in a radial direction at most up to a distance greater than zero from an edge of the lens.

### BRIEF DESCRIPTION OF THE DRAWINGS

The present invention will be described in even greater detail below based on the exemplary figures. The invention is not limited to the exemplary embodiments. All features described and/or illustrated herein can be used alone or combined in different combinations in embodiments of the invention. The features and advantages of various embodiments of the present invention will become apparent by reading the following detailed description with reference to the attached drawings which illustrate the following:

FIG. 1 is a schematic front view of an eye with a lens which is divided into several segments and, in one of the segments, is finely fragmented according to an embodiment of the invention;

FIG. 2 is a schematic front view of an eye with a lens which is segmented and finely fragmented according to a further embodiment of the invention;

FIG. 3 is a schematic front view of an eye with a lens which is segmented and finely fragmented according to a further embodiment of the invention;

FIG. 4 is a schematic front view of an eye with a lens which is segmented and finely fragmented according to a further embodiment of the invention; and

FIG. 5 is a schematic representation of the structure of a control device of a laser system according to an embodiment of the invention.

### DETAILED DESCRIPTION

An embodiment of the present invention provides a device and a method with which a lens of a patient's eye can be fragmented, in particular by control of a laser, such that removal can be performed in a convenient manner, preferably with low radiation exposure of the patient's eye.

An embodiment of the invention provides a control device for a laser system with a laser for introducing cuts into a lens of a human or animal eye, which is set up to arrange and move a focal point of a laser beam of the laser within the lens, wherein the focal point can (first) be moved for at least one (rough) cut for the fragmentation and/or segmentation of the lens into individual areas, in particular fragments or segments, and (then) can be moved according to a definable cutting pattern (of several fine cuts or individual laser spots) for the fine fragmentation of the lens; wherein the control device is set up to determine an individual one of the fragments and/or segments and, for the fine fragmentation within the determined segment, to move the focal point in a radial direction at most up to a distance greater than zero from an edge of the lens, in particular up to at least one of the segment cuts.

The cutting pattern for the fine fragmentation is placed only on a small part of the lens and also only on a part of the (respective) fragment or segment, in particular only on an individual nuclear segment. The total energy input can hereby be minimized. The focal point can be moved for the segmentation of at least the nucleus of the lens or additionally a cortex of the lens. The cutting pattern for the fine fragmentation can be finely selected such that the corresponding fragment or segment, in particular nuclear segment, can be removed without further phacoemulsification and an application of ultrasound energy onto the corresponding fragment or segment can be dispensed with.

It is not necessary to finely fragment the entire (respective) fragment or segment using the laser. Instead, it is sufficient to limit the fine fragmentation to an area (in particular an area of the fragment or segment) in which the lens is particularly hard, in particular to the area of the lens nucleus and/or an area which corresponds at least approximately to the outer diameter of a distal end of a phacoemulsification probe. The at least one segment cut is preferably a radial cut, i.e. an in particular rectilinear cut aligned in a radial direction.

The control apparatus can be set up to move the focal point in relation to a central point of the lens by an amount smaller than the radial extent of the lens. For this the control apparatus preferably has an arrangement by means of which the radius or the radial extent of the lens, as well as the radial position of the focal point in relation to a central point of the lens can be taken into account. The focal point can be moved in a radial direction by an amount smaller than a radius of the lens and maintained at a distance >0 from the edge of the lens.

The control apparatus can be set up to capture a radial extent of the lens (a radius of the lens) as well as an individual one of the segments and, for the fine fragmentation in the individual segment, in particular nuclear segment, to move the focal point within the segment (nuclear segment) in a radial direction in relation to a central point of the lens at most by an amount smaller than the radial extent of the lens.

The control apparatus need not necessarily capture the radial dimensions of the lens in the sense of an optical measurement, but rather the dimensions of the lens can also be determined from geometrical data (in particular stored in a memory of the control apparatus), or they can be transmitted to the control apparatus from an external device. The radial dimensions include in particular an edge of the lens, but also a central point of the lens, since the radial position of the focal point can be easily set starting from a central point of the lens. The control apparatus can also perform a movement within definable radial boundary values, with which it can be ensured, independently of a respective patient, that e.g. only the lens nucleus is finely fragmented. The radial boundary value is then e.g. smaller than the average radius of a lens nucleus of a patient of a particular age or with a particular background. The same applies to the individual segment. The control apparatus can e.g. perform as standard a division into equally large segments in the shape of cake slices, e.g. four or six segments, wherein the segment cuts can be arranged in a definable manner, e.g. relative to an access cut into the eye. Thus the boundaries of a respective fragment or segment also need not necessarily be captured optically. Preferably, the cuts are introduced such that as little energy as possible is required, in particular two rectilinear or flat segment cuts, which are aligned orthogonal to each other and divide the lens into four segments with a circular cross section.

By a segmentation is meant a division or rough fragmentation of the lens into several segments, thus the introduction of one or more (in particular rough) cuts, which make it possible to divide the lens into several segments. By a fragmentation is meant a division or rough fragmentation of the lens into several fragments, thus not entirely delimited areas. In this case, one or more (in particular rough) cuts are introduced, which make it possible to divide the lens into several fragments. A rough cut can be e.g. a cut which passes through the lens in its entirety, be it in an axial or a radial direction. Preferably, the lens is broken up into several individual parts. A segmentation can be effected e.g. along main axes of the lens, or along planes arranged diagonally in relation to main axes of the lens. The segmentation can be effected e.g. using lasers, which emit nano-, pico- or femtosecond pulses and cause ablative or disruptive tissue interactions due to a multi-photon absorption.

By a fine fragmentation is meant an introduction of such fine cuts or cuts arranged so close to each other that the corresponding part of the lens is softened and can be suctioned off. Cuts are not necessarily required, but rather the fine fragmentation can also be achieved using individual spots or other laser energy introduced in a punctiform manner, be it alone or in combination with cuts. Preferably, the fine fragmentation is likewise effected using pulsed laser radiation, which causes ablative or disruptive tissue interactions via multi-photon absorption.

By setting up the control apparatus to allow the fine fragmentation to be carried out up to at least one of the segment cuts, it can be ensured that an aspiration probe can easily penetrate the lens nucleus, in particular at the interface of the individual segments. According to a variant, the individual segment is finely fragmented up to all segment cuts delimiting the segment. The individual segment can hereby be suctioned off in its entirety and isolated from the other segments. It can be sufficient to provide a segment in the size of a distal end of the aspiration probe, with the result that as little tissue as possible has to be finely fragmented in order to create space in the lens.

The control device can be equipped with the following components:
a focus-measuring apparatus which is set up to determine a distance of a focal point of a pulsed laser beam of the laser in relation to a reference point of the laser; and
an optical capture unit which is set up to capture a position and/or geometry of the tissue relative to the reference point.

According to an embodiment, the control device is set up to capture an edge and/or central point of the lens and, for the fine fragmentation in the individual segment, in particular nuclear segment, to move the focal point within the segment (nuclear segment) at most up to a definable radial distance from the edge and/or central point. The relation to the edge or central point of the lens can ensure that the focal point can be arranged in a determined radial position in a precise manner and is not moved radially outwards further than a determined radial boundary value. In other words, the control apparatus is set up to move the focal point in at least one of the nuclear segments at a radial distance from a central point of the lens which is smaller than or equal to the radius of the lens nucleus.

According to an embodiment, the control device is set up to capture or to take into account a radius of the nucleus, wherein the radial distance is set as greater than or equal to or at least approximately equal to a distance between the edge and the nucleus. As the radius of the nucleus is captured or taken into account, the radial extent of the lens cortex can be determined or taken into account indirectly. Here, an energy input can be restricted to the area of the lens tissue which is the hardest. The fine fragmentation can be effected without the lens cortex being (in all likelihood unnecessarily) loaded with energy. This makes it possible to further minimize the energy introduced into the eye. The radial distance can also be exactly equal to the distance between the nucleus and the edge.

The lens is formed by a lens nucleus and a lens cortex, wherein by lens cortex is preferably meant here the entire area radially outside the lens nucleus. Here, the distance between the nucleus and the edge which can be measured or seen by an optical capture system or an optics apparatus corresponds by definition to the lens cortex. The lens cortex is the area radially outwards from the nucleus, between the nucleus and the edge of the lens.

The radius of the nucleus can be determined visually e.g. by the operator on the basis of image acquisitions, tomographic acquisitions by means of optical coherence tomography (OCT) or ultrasonic tomography, or by means of a Scheimpflug camera.

According to a variant, the control device is set up to determine an (imaginary) separation surface, which delimits the lens nucleus from the lens cortex, wherein, for the fine fragmentation in an individual nuclear segment, the focal point can be arranged and moved within the nuclear segment up to the separation surface, in particular in a radial direction at most up to the separation surface. It is hereby possible to divide and finely fragment selectively only the nucleus. The nucleus, as the hardest part of the lens, can be segmented in a convenient manner, without also having to segment the lens cortex in the same way, in particular having to segment more than is required. The energy input into the eye can hereby be (further) minimized, and a patient can be treated in a gentle manner. The separation surface can also represent a transition area, in particular an annular area, which lies between a clearly identifiable area of the lens cortex and a clearly identifiable area of the lens nucleus.

In the case of a fine fragmentation within the separation surface, the lens can be fragmented in an energetically optimized manner, and space can be efficiently created in the lens, in particular in order to be able to introduce a free distal end of an aspiration probe into the lens. By separation surface is meant a surface or an area which marks the transition from the lens nucleus to the lens cortex.

According to an embodiment, for the fine fragmentation in two bordering segments of nucleus, the control device is set up to move the focal point within the nucleus. Space can hereby be created in an area within the lens which has already been passed through by at least one rough cut or segment cut. This can ensure, irrespective of the accuracy of the fine fragmentation, that the operator can introduce the distal end of an aspiration probe into the lens without difficulty, namely in the area of the rough cut, and can begin to suction off the nucleus of the lens. Preferably, each nuclear segment is finely fragmented only to a certain extent.

According to an embodiment, the radial distance is set as greater than the distance between the edge and the nucleus. The energy introduced can hereby be further reduced. In other words, the control device is set up, for the fine fragmentation within the nucleus, to move the focal point at a definable radial distance from an (imaginary) separation surface lying between the nucleus and the lens.

By means of a distance from the separation surface it can be ensured that only the area of the lens nucleus which is the hardest and most difficult to remove is loaded with energy. As a distance from the separation surface is maintained, as much as possible of the energy expended can be used for the hardest part of the lens nucleus.

According to an embodiment, the control device is set up, for the fine fragmentation within the nucleus, to move the focal point in a central area of the nucleus (irrespective of the arrangement of the individual segment relative to the central area). Hereby, a fine fragmentation can also be provided in a central area of the nucleus irrespective of the position and geometry of the previously formed segments, wherein the fine fragmentation is in particular provided with a geometry which is partially cylindrical (in the central area) and in the shape of circular or spherical segments (in a circular or spherical segment). In this way, space can be created for the distal end of an aspiration probe such that the distal end can be guided to all segments. For the fine fragmentation, the distal end can be led radially inwards to each individual segment starting from the central area. Alternatively, a phacoemulsification probe can also be positioned in the central area.

The central area is preferably connected to the finely fragmented nuclear segment or overlaps the finely fragmented nuclear segment. The central area preferably extends concentrically around a central point of the lens or of the nucleus. The central point can relate to a centre of the front or rear casing surface or end face of the lens, or a centre of the volume of the lens. Preferably, the focal point can be moved such that both a partially cylindrical fine fragmentation and a fine fragmentation in the shape of circular or spherical segments are formed. According to a variant, a fine fragmentation of the entire nuclear segment is effected.

According to an embodiment, for the segmentation, the at least one cut is made through the central area. A respective segment can hereby be made easily accessible. Each segment protrudes partially into the central area, and space can be created in each segment by means of the fine fragmentation of the central area, in order to then remove the segment by aspiration alone or in conjunction with phacoemulsification.

An embodiment of the invention provides a medical laser system with a laser and with a control device, wherein the control device has at least one actuator coupled to the laser and is set up to move a focal point of a laser beam of the laser by means of the at least one actuator. The advantages already described in connection with the control device hereby result.

An embodiment of the invention provides a method for controlling a laser system having a laser for introducing cuts into a lens of a human or animal eye, with the steps of:
a) moving a focal point of a pulsed laser beam of the laser for at least one cut to divide at least a nucleus or additionally a cortex of the lens into individual fragments and/or segments;
b) moving the focal point according to a definable cutting pattern for the fine fragmentation of the lens.

For the fine fragmentation within an individual one of the fragments and/or segments, the focal point is moved in a radial direction at most up to a distance greater than zero from an edge of the lens. The advantages already described in connection with the control device result from such a method. A radial extent of the lens, as well as an individual one of the segments, can be captured, and in step b) the focal point in the individual segment, in particular nuclear segment, can be moved within the segment (nuclear segment) in a radial direction by an amount smaller than the radial extent of the lens.

Preferably, an edge and/or central point of the lens is captured and in step b) the focal point in an individual segment, in particular nuclear segment, is moved within the segment (nuclear segment) up to a definable radial distance from the edge or central point. According to a variant, a determined radial distance can also be maintained by determining an (imaginary) separation surface which delimits the lens nucleus from the lens cortex and, during the fine fragmentation exclusively in an individual nuclear segment, moving the focal point within the nuclear segment, wherein the focal point is moved in a radial direction at most up to the separation surface. The separation surface extends in a radius around the central point of the lens, which at least approximately corresponds to the radius of the lens nucleus.

The method can furthermore have the following steps of: determining a distance of a focal point of a pulsed laser beam of the laser in relation to a geometrical reference point of the laser or a reference mark introduced in another way (e.g. a mark implemented in a patient interface or a reference surface in a contact lens); and capturing a position or geometry of the tissue relative to the reference point and arranging the focal point within the lens relative to the reference point. Hereby, the laser can also be controlled in relation to a reference point preferably calibrated before a respective treatment.

An embodiment of the invention provides a computer program product including code, which is set up to carry out a method on a computer or an arithmetic unit. An embodiment of the invention also provides a storage medium, on which such a computer program product is stored.

An embodiment of the invention provides a method for introducing cuts into a lens of a human or animal eye by means of a laser, with the steps of:
1) moving a focal point of a pulsed laser beam of the laser such that, for at least one cut, a nucleus or additionally a cortex of the lens is (roughly) divided into several segments, in particular into four quadrants; and,
2) for the fine fragmentation within an individual one of the segments, moving the focal point in a radial direction at most up to a distance greater than zero from an edge of the lens.

The segments are preferably quadrants or six equal portions of a circle projected onto the lens or eight equal portions of a circle projected onto the lens. Particularly preferably, the segment is a circle, preferably a circle with a central point approximately in the central point of the lens. Particularly preferably, the segments are an inner circle and adjoining segments of the remaining annulus of the lens broken up into four, six or eight equal portions without the inner circle.

In step 2) a capture of a radial extent of the lens, as well as of an individual one of the segments, and a movement of the focal point in the individual segment, in particular nuclear segment, can be effected within the segment in a radial direction in relation to a central point of the lens by an amount smaller than the radial extent.

The movement in a radial direction can be effected up to a separation surface, which delimits the lens nucleus from the lens cortex, in particular as a function of the distance and of the position and/or geometry.

The method can furthermore have the following steps of: determining a distance of the focal point in relation to a reference point of the laser; and capturing a position and/or geometry of the lens relative to the reference point and arranging the focal point within the lens relative to the reference point. The movement can be effected here as a function of the distance and of the position and/or geometry.

The segment is preferably finely fragmented only in an area which is at least approximately as large as the distal end of an aspiration or phacoemulsification probe used, preferably up to 3 mm, particularly preferably up to 2 mm, in particular preferably up to 1 mm. The energy input can hereby be further minimized. Only as much space as is required for introducing the phacoemulsification probe is created in the lens nucleus.

A rough fragment or segment of the lens nucleus can be finely fragmented, which is easier to achieve starting from the access cut. Preferably, the finely fragmented area lies opposite an access cut into the lens.

An embodiment of the invention provides a method for removing a lens from a human or animal eye, with the steps of:
A) segmenting or roughly fragmenting at least a nucleus or additionally a cortex of the lens by means of a laser such that at least the lens nucleus is divided into several segments;
B) finely fragmenting one of the nuclear segments by means of the laser within the nuclear segment in a radial direction by an amount smaller than the radial extent of the lens, such that the finely fragmented nuclear segment can be suctioned off;
C) positioning a free end of an aspiration probe in the finely fragmented nuclear segment;
D) suctioning off the finely fragmented nuclear segment, in particular irrespective of the lens cortex in the segment; and
E) suctioning off the lens cortex at least partially, in particular segmentally.
   Here, a part of the lens can be suctioned off first, and then, if there is already space within the lens for manoeuvring with the aspiration handpiece, other parts of the lens can be further processed and suctioned off. The rough fragmentation is preferably effected by introducing two cuts, in particular two cuts arranged orthogonal to each other, which divide the lens into four segments, in particular four quadrants. Step B) is preferably effected, for the fine fragmentation within the segment, by moving the focal point in a radial direction at most up to a distance greater than zero from an edge of the lens.

In FIG. 1 an eye 20 is shown schematically, in which a lens 21 is arranged behind an iris 24 which surrounds a pupil 23. The lens 21 has a lens nucleus 21a and a lens cortex 21b. A boundary extending at a determined radial distance around the lens nucleus 21a or a transition between the lens nucleus 21a and the lens cortex 21b is marked by an (imaginary) separation surface T indicated schematically. The separation surface T marks an edge of the nucleus 21a. The lens 21 has an edge R, which is concealed by the iris 24. A distance r between the nucleus 21a and the edge R marks the area occupied by the lens cortex 21b.

The lens nucleus 21a is divided by two rough cuts 21.1, which are arranged diagonally in relation to a main axis (here the vertical axis) of the lens 21 and divide the lens 21 into four fragments and/or segments 21.1a. An individual segment 21.1a of the lens nucleus 21a is finely fragmented in its entirety, but not the part of the segment occupied by the lens cortex 21b. However, a fine fragmentation 21.2 is provided not only in this segment 21.1a, but also in a central area of the nucleus 21a. The central area extends in particular concentrically around a central point M of the lens 21. An aspiration probe (not represented) can be guided to the nucleus 21a of the lens 21 via an access cut 24.1. The access cut 24.1 is arranged in a position which can be easily reached by a medical instrument, in particular the tip of an aspiration handpiece.

By means of a fine fragmentation 21.2, which is provided both in only one individual segment 21.1a and in a central area of the nucleus 21a, the segments 21.1a can be separated from each other well, and a probe for aspiration, and alternatively also for phacoemulsification, can be easily led to each of the three remaining segments 21.1a after removal of the finely fragmented areas, in particular starting from the middle or the central area of the nucleus 21a. By means of this fine fragmentation 21.2 which is partially cylindrical and in the shape of circular or spherical segments, space can be created in the lens nucleus 21a without introducing much energy into the eye. Only the hardest and most easily accessible area of the lens nucleus 21a is finely fragmented. This makes a gentle treatment possible.

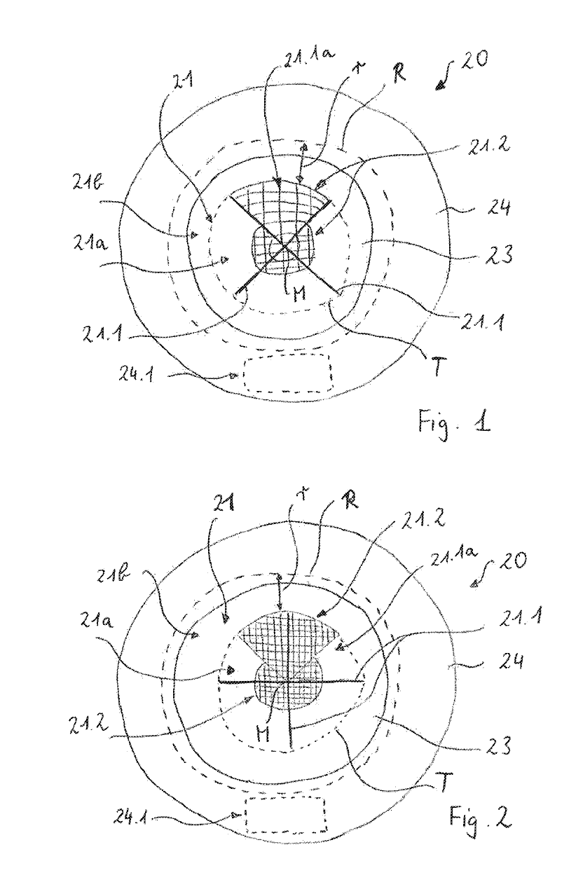

An eye 20 with a structure comparable to the one in FIG. 1 is shown in FIG. 2. The lens nucleus 21a is divided in this case into two rough cuts 21.1, which are arranged perpendicularly in relation to a main axis (here the vertical axis) of the lens 21 on the one hand and in the main axis on the other hand, and divide the lens 21 into four segments 21.1a. As in the embodiment example of FIG. 1, only the lens nucleus 21a is segmented. A fine fragmentation 21.2 is provided in two segments of the four segments 21.1a, wherein the fine fragmentation 21.2 is provided only in a subarea of the two segments 21.1a in each case. In this arrangement of the fine fragmentation 21.2, it can be ensured that the entire tissue is finely fragmented at the rough cut 21.1 on the boundary surface between two segments 21.1a. Both segments 21.1a can hereby be easily suctioned off successively.

The fine fragmentation 21.2 is (as already described in connection with FIG. 1) also provided in a central area of the nucleus 21a, wherein a fine fragmentation 21.2 which is partially cylindrical and in the shape of circular or spherical segments is provided. The central area extends in particular concentrically around a central point M of the lens 21.

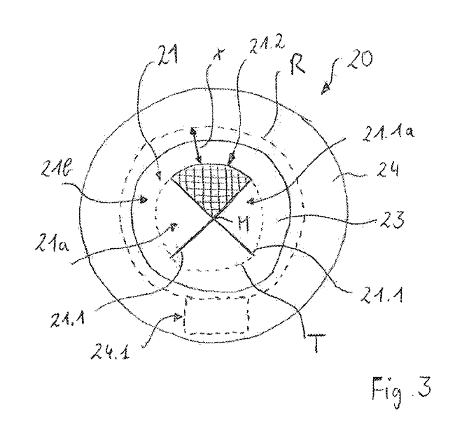

An embodiment example in which an additional fine fragmentation of a central area of the lens nucleus 21a is not effected is shown in FIG. 3. The lens nucleus 21a is divided into two rough cuts 21.1 which pass through a central point M of the lens 21, are arranged diagonally in relation to a main axis (here the vertical axis) of the lens 21 and divide the lens 21 into four segments 21.1a. An individual segment 21.1a of the lens nucleus 21a is finely fragmented in its entirety. A fine fragmentation 21.2 is provided exclusively in this segment 21.1a.

In particular in the case of a lens nucleus 21a which is not particularly hard, e.g. in the case of younger patients, this type of fine fragmentation 21.2 may be sufficient. In other words, it is not strictly necessary to additionally also finely fragment the central area of the lens nucleus 21.2 shown in FIGS. 1 and 2.

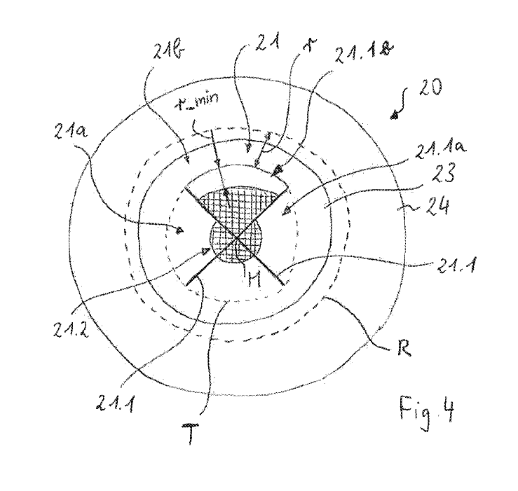

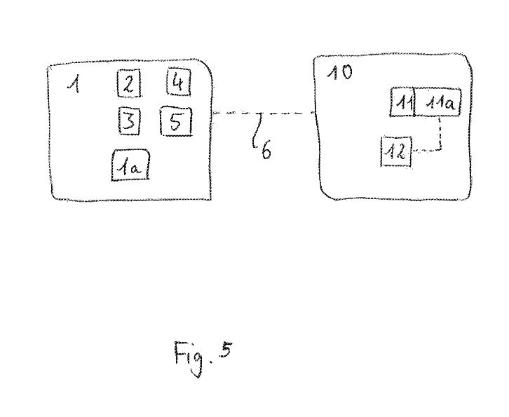

A variant of the embodiment example shown in FIG. 1 is shown in FIG. 4. The lens nucleus 21a is again divided by two rough cuts 21.1, which are arranged diagonally in relation to a main axis (here the vertical axis) of the lens 21 and divide the lens 21 into four segments 21.1a. An individual segment 21.1a of the lens nucleus 21a is partially finely fragmented. The fine fragmentation 21.2 is (as already described in connection with FIG. 1) also provided in a central area of the nucleus 21a, wherein a fine fragmentation 21.2 with a geometry which is partially cylindrical and in the shape of circular or spherical segments is provided. The central area extends in particular concentrically around a central point M of the lens 21.

In addition, in this embodiment example, an unfragmented area 21.1b of the nuclear segment 21.1a, which is not finely fragmented, is also provided radially outwards from the fine fragmentation 21.2. The unfragmented area 21.1b extends outwards at the edge of the lens nucleus 21a at a predetermined minimum distance r_min from the separation surface T. The minimum distance r_min is preferably 5 to 95 percent, in particular preferably 20 to 85 percent, particularly preferably more than 50%, in particular preferably 50 to 95%, preferably 60 to 90% of a radius R, which the lens nucleus 21a has in this area.

In FIG. 5 a control device 1 is shown which has a focus-measuring apparatus 2, an optical capture unit 3, an arithmetic unit 4 as well as a storage medium 6. The control device 1 is connected to a laser system 10 via a communication interface 6. The communication interface 6 can be designed wireless and/or wired. The laser system 10 has a laser 11, in particular a nano-, pico- or femtosecond laser, with an optics apparatus 11a as well as one or more actuators 12. The control device 1 has at least one actuator 1a, which is set up to actuate the laser 11 either directly or in active connection with the at least one actuator 12 of the laser 11.

The control device 1 is set up to control the actuator(s) 1a; 12 such that, during the fine fragmentation of a lens exclusively in an individual nuclear segment of a lens nucleus, a focal point of a laser beam of the laser 11 is arranged and moved within the nuclear segment, wherein the focal point is moved in a radial direction at a definable radial distance from the central point and/or edge of the lens, in particular at most up to a separation surface which delimits the lens nucleus from the lens cortex.

The energy introduced into the eye can be minimized by means of a fine fragmentation of only an individual one of the segments. By removal of the finely fragmented area of the lens by aspiration/irrigation and phacoemulsification, as much space as is required in order to be able to easily carry out the removal of the remaining lens parts is created in the lens of the eye.

While the invention has been illustrated and described in detail in the drawings and foregoing description, such illustration and description are to be considered illustrative or exemplary and not restrictive. It will be understood that changes and modifications may be made by those of ordinary skill within the scope of the following claims. In particular, the present invention covers further embodiments with any combination of features from different embodiments described above and below.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

### LIST OF REFERENCE NUMBERS

1 Control device
1a Actuator
2 Focus-measuring apparatus
3 Optical capture unit
4 Arithmetic unit
5 Storage medium
6 Connection between control device and laser system
10 Laser system
11 Femtosecond laser
11a Optics apparatus
12 Actuator
20 Eye
21 Lens
21a Lens nucleus
21b Lens cortex
21.1 Rough cut
21.1a Individual roughly divided area, in particular individual fragment or segment
21.1b Unfragmented area of the fragment or segment, which is not finely fragmented
21.2 Fine fragmentation or cutting pattern
23 Pupil
24 Iris
24.1 Access cut
r Distance between an edge of the lens and the nucleus of the lens
r_min Predetermined minimum distance from the edge of the nucleus
M Central point of the lens
R Edge of the lens
T Separation surface

1. A control device for a laser system having a laser for introducing cuts into a lens of a human or animal eye, the control device being configured to:
arrange and move a focal point of a laser beam of the laser within the lens, wherein the focal point can be moved for at least one cut for the fragmentation or segmentation of the lens into individual fragments or segments and can be moved according to a definable cutting pattern for the fine fragmentation of the lens;
determine an individual one of the segments; and
move, for the fine fragmentation within the determined segment, the focal point in a radial direction at most up to a distance greater than zero from an edge of the lens.

2. The control device according to claim 1, further configured to capture the edge or a central point of the lens and,
move, for the fine fragmentation within the segment, the focal point up to a definable radial distance from the edge or central point.

3. The control device according to claim 2, further configured to capture a radius of a nucleus of the lens, wherein the radial distance is set as greater than or equal to a distance between the edge and the nucleus.

4. The control device according to claim 3, wherein the radial distance is set as greater than the distance between the edge and the nucleus.

5. The control device according to claim 1, further configured to move, for the fine fragmentation within the nucleus, the focal point in a central area of the nucleus.

6. The control device according to claim 5, wherein for the fragmentation or segmentation, the at least one cut is made through the central area.

7. A medical laser system comprising:
a laser; and
a control device configured to arrange and move a focal point of a laser beam of the laser within the lens, wherein the focal point can be moved for at least one cut for the fragmentation or segmentation of the lens into individual fragments or segments and can be moved according to a definable cutting pattern for the fine fragmentation of the lens, determine an individual one of the segments, and move, for the fine fragmentation within the determined segment, the focal point in a radial direction at most up to a distance greater than zero from an edge of the lens according to one of the previous claims, the control device comprising:
at least one actuator coupled to the laser and configured to move a focal point of a laser beam of the laser by means of the at least one actuator.

8. A method for controlling a laser system having a laser for introducing cuts into a lens of a human or animal eye, the method comprising:
a) moving a focal point of a pulsed laser beam of the laser for at least one cut to divide the lens into individual fragments or segments; and
b) moving the focal point according to a definable cutting pattern for the fine fragmentation of the lens;
wherein for the fine fragmentation within an individual one of the fragments or segments, the focal point is moved in a radial direction at most up to a distance greater than zero from an edge of the lens.

9. A computer program comprising code including instructions for:
a) moving a focal point of a pulsed laser beam of the laser for at least one cut to divide the lens into individual fragments or segments; and
b) moving the focal point according to a definable cutting pattern for the fine fragmentation of the lens;
wherein for the fine fragmentation within an individual one of the fragments or segments, the focal point is moved in a radial direction at most up to a distance greater than zero from an edge of the lens.

10. A computer-readable storage medium having stored thereon computer executable instructions for:
a) moving a focal point of a pulsed laser beam of the laser for at least one cut to divide the lens into individual fragments or segments; and
b) moving the focal point according to a definable cutting pattern for the fine fragmentation of the lens;
wherein for the fine fragmentation within an individual one of the fragments or segments, the focal point is moved in a radial direction at most up to a distance greater than zero from an edge of the lens.
